(19) United States
(12) Patent Application Publication
(10) Pub. No.: US 2014/0005516 A1
(43) Pub. Date: Jan. 2, 2014

(54) DIAGNOSIS IMAGE APPARATUS AND OPERATION METHOD THEREOF

(71) Applicant: SAMSUNG MEDISON CO., LTD., Gangwon-do (KR)

(72) Inventors: Bong-Heon LEE, Gangwon-do (KR); Jin-yong LEE, Gangwon-do (KR); Sung-wook PARK, Gangwon-do (KR)

(21) Appl. No.: 13/924,120

(22) Filed: Jun. 21, 2013

(30) Foreign Application Priority Data
Jun. 28, 2012 (KR) 10-2012-0070231

(51) Int. Cl. A61B 5/02

(52) U.S. Cl. CPC A61B 5/02
USPC 600/407

(57) ABSTRACT

A diagnosis imaging apparatus and an operation method thereof are provided. The diagnosis imaging apparatus includes: an image processing apparatus for acquiring a first image including a first contour of a heart wall based on first image data of a heart of a subject acquired when the subject is subjected to a first stress intensity level, acquiring a second image including a second contour of the heart wall based on second image data of the heart of the subject acquired when the subject is subjected to a second stress intensity level, and acquiring a third image representing a difference between the first contour and the second contour based on the first image and the second image; and a display apparatus for displaying the third image.

### CROSS-REFERENCE TO RELATED PATENT APPLICATION

This application claims the benefit of Korean Patent Application No. 10-2012-0070231, filed on Jun. 28, 2012, in the Korean Intellectual Property Office, the disclosure of which is incorporated herein in its entirety by reference.

### BACKGROUND OF THE INVENTION

1. Field of the Invention

The present invention relates to a diagnosis imaging apparatus and an operation method thereof.

2. Description of the Related Art

Diagnosis imaging apparatuses refer to medical imaging apparatuses such as ultrasound imaging apparatuses, computed tomography (CT) apparatuses, or magnetic resonance imaging (MRI) apparatuses.

A diagnosis imaging apparatus may acquire a first image of the heart of a subject when the subject is subjected to low intensity stress, and acquire a second image of the heart when the subject is subjected to high intensity stress. A user may diagnose if the subject has an illness in the heart by comparing the first image with the second image. This is because changes in cardiac muscle thickness when the subject is subjected to stress at different intensity levels depend on whether the subject has an illness in the heart or not.

However, when the user diagnoses an illness by separately viewing the first and second images, the user may not readily perceive a difference between the first and second images during the diagnosis, which may reduce diagnosis accuracy. To increase the diagnosis accuracy, the user may need to perform an additional task, such as reconfirming the first and second images. However, the additional task may delay the diagnosis time.

Therefore, there is a demand for an efficient diagnosis imaging apparatus and an operation method thereof.

### SUMMARY OF THE INVENTION

The present invention provides an efficient diagnosis imaging apparatus and an operation method thereof.

According to an aspect of the present invention, there is provided a diagnosis imaging apparatus including: an image processing apparatus for acquiring a first image including a first contour of a heart wall based on first image data of a heart of a subject acquired when the subject is subjected to a first stress intensity level, acquiring a second image including a second contour of the heart wall based on second image data of the heart of the subject acquired when the subject is subjected to a second stress intensity level, and acquiring a third image representing a difference between the first contour and the second contour based on the first image and the second image; and a display apparatus for displaying the third image.

According to another aspect of the present invention, there is provided an operation method of a diagnosis imaging apparatus, the method including: acquiring a first image including a first contour of a heart wall based on first image data of a heart of a subject acquired when the subject is subjected to a first stress intensity level; acquiring a second image including a second contour of the heart wall based on second image data of the heart of the subject acquired when the subject is subjected to a second stress intensity level; acquiring a third image representing a difference between the first contour and the second contour based on the first image and the second image; and displaying the third image.

According to another aspect of the present invention, there is provided a display apparatus displaying a third image representing a difference between a first contour and a second contour of a heart wall, the first contour of the heart wall being based on first image data of a heart of a subject acquired when the subject is subjected to a first stress intensity level, and the second contour being based on second image data of the heart of the subject acquired when the subject is subjected to a second stress intensity level.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram of a diagnosis imaging apparatus according to an embodiment of the present invention;

FIG. 2 illustrates a first image and a second image acquired by an image processing apparatus of FIG. 1, according to an embodiment of the present invention;

FIG. 3 illustrates a third image displayed on a display apparatus of FIG. 1, according to an embodiment of the present invention;

FIG. 4 illustrates a third image displayed on the display apparatus of FIG. 1, according to another embodiment of the present invention;

FIG. 5 is a view illustrating changes in thickness of a cardiac muscle according to types of heart conditions and stress levels;

FIG. 6 illustrates first to third images displayed on the display apparatus of FIG. 1, according to another embodiment of the present invention;

FIG. 7 illustrates first to third images displayed on the display apparatus of FIG. 1, according to another embodiment of the present invention;

FIG. 8 illustrates first to fourth images displayed on the display apparatus of FIG. 1, according to another embodiment of the present invention;

FIG. 9 illustrates first to third images displayed on the display apparatus of FIG. 1, according to another embodiment of the present invention; and

FIG. 10 is a flowchart of an operation method of a diagnosis imaging apparatus, according to an embodiment of the present invention.

### DETAILED DESCRIPTION OF THE INVENTION

As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments of the present invention are described in detail with reference to the appended drawings.

FIG. 1 is a block diagram of a diagnosis imaging apparatus 100 according to an embodiment of the present invention.

Referring to FIG. 1, the diagnosis imaging apparatus 100 includes an image processing apparatus 110 and a display apparatus 120. The diagnosis imaging apparatus 100 may further include a storing apparatus 130 and an input apparatus 140. A subject 200 may be an animal body, including a human body, having a heart 210.

The diagnosis imaging apparatus 100 is an apparatus for diagnosing whether the subject 200 has an illness in the heart 210, based on image data of the heart 210 of the subject 200. In some embodiments, the diagnosis imaging apparatus 100 may be a medical imaging apparatus, such as an ultrasound imaging apparatus, a computed tomography (CT) apparatus, or magnetic resonance imaging (MRI) apparatus.

The image processing apparatus 110 may acquire first image data of the heart 210 of the subject 200 when the subject 200 is subjected to a first stress intensity level, and may acquire second image data of the heart 210 when the subject 200 is subjected to a second stress intensity level higher than the first intensity level. The first image data and the second image data are obtained by subjecting the subject 200 to different stress intensity levels. The first image data and second image data may be 2-dimensional (2D) image data or 3-dimensional (3D) volume data, but are not limited thereto. The storing apparatus 130 may store the first image data and the second image data.

The intensity level of stress may be controlled by adjusting the intensity of exercise the subject 200 does or a dose of drug administered to the subject 200. For example, the first image data may be image data acquired from the subject 200 to which a first drug dosage is administered in a relaxed state, and the second image data may be image data acquired from the subject 200 to which a second drug dosage higher than the first drug dosage is administered in a relaxed state. The drug administered to the subject 100 may be dobutamine. In some other embodiments, the first image data may be image data acquired while the subject 200 is exercising at a first exercise intensity level, and the second image data may be image data acquired while the subject 200 is exercising at a second intensity level higher than the first exercise intensity level. For example, the exercise at the first intensity level may be riding a bike, and the exercise at the second intensity level may be walking or running.

The image processing apparatus 110 may acquire a first image including a first contour of the heart wall of the heart 210 based on the first image data, and acquire a second image including a second contour of the heart wall of the heart 210 based on the second image data.

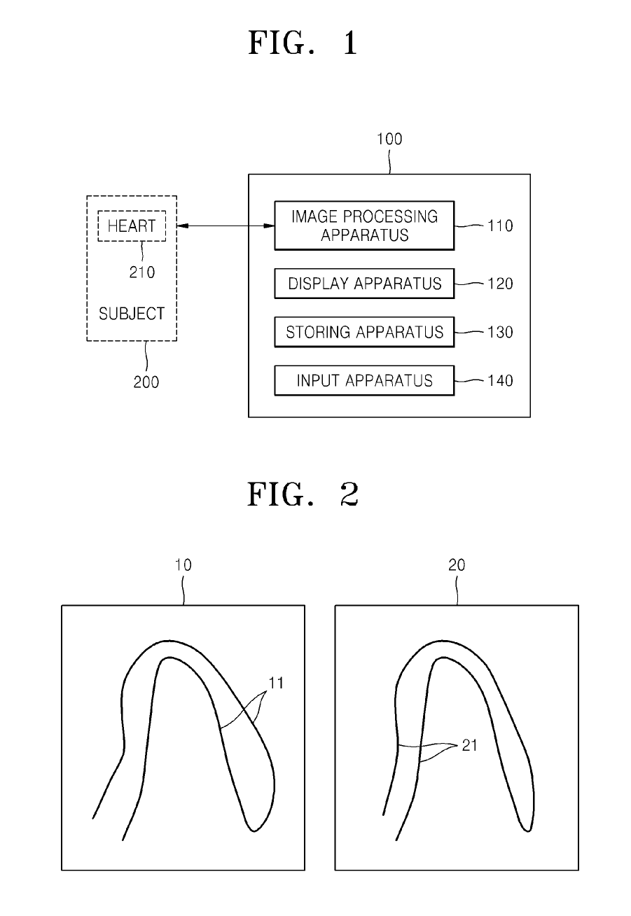

FIG. 2 illustrates a first image 10 and a second image 20 acquired by the image processing apparatus 110 of FIG. 1, according to an embodiment of the present invention. Referring to FIGS. 1 and 2, the first image 10 may include a first contour 11 of the heart wall of the heart 210 when the subject 200 is subjected to a first stress intensity level. The second image 21 may include a second contour 21 of the heart wall of the heart 210 when the subject 200 is subjected to a second stress intensity level higher than the first stress intensity level.

The first image 10 may be an image including the first contour 11 of the heart wall based on a cross-sectional image of the heart 210 acquired from the first image data, and the second image 20 may be an image including the second contour 21 of the heart wall based on a cross-sectional image of the heart 210 acquired from the second image data. The cross-sectional image of the heart 210 may be a 4-chamber view, a 3-chamber view, or a 2-chamber view. Each of the first contour 11 and the second contour 21 may represent the heart wall of the left ventricle.

Although in FIG. 2 the first image 10 and the second image 20 are illustrated as only including the first contour 11 and the second contour 21, respectively, the embodiment is not limited thereto. In some other embodiments, the first image 10 and the second image 20 may be images with the first contour 11 and the second contour 21 on cross-sections of the heart, respectively.

The first contour 11 and the second contour 21 may be automatically or manually drawn on the first image 10 and the second image 20, respectively. For example, the first contour 11 and the second contour 21 may be automatically drawn based on brightness. In some other embodiments, the first contour 11 and the second contour 21 may be drawn based on a user input via the input apparatus 140. The input apparatus 140 may be, for example, a touch panel on the display apparatus 120. A user may draw contours of the heart wall on a displayed cross-sectional image of the heart by using the input apparatus 140, so that the first contour 11 and the second contour 21 may be included in the first image and the second image 20, respectively.

The image processing apparatus 110 may acquire a third image representing a difference between the first contour 11 and the second contour 21 based on the first image 10 and the second image 20. The display apparatus 120 may display the third image.

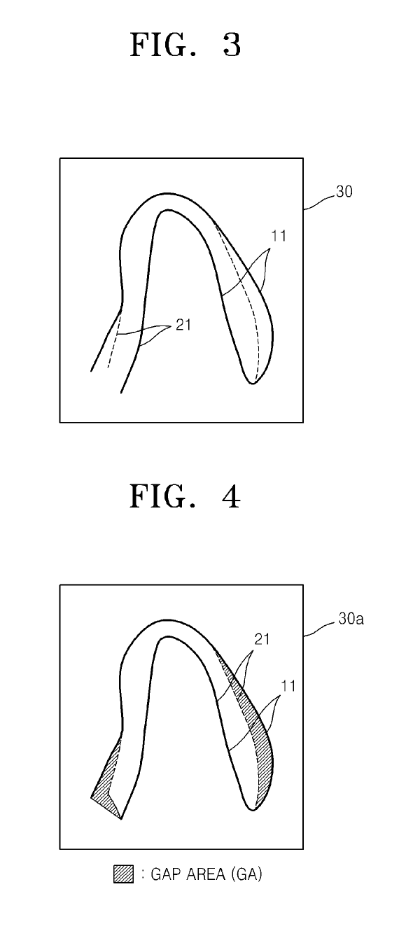

FIG. 3 illustrates a third image 30 displayed on the display apparatus 120 of FIG. 1, according to another embodiment of the present invention.

Referring to FIGS. 2 and 3, the first image 10 including the first contour 11 and the second image 20 including the second contour 21 are overlapped on the third image 30 so that a difference between the first contour 11 and the second contour 21 is represented on the third image 30. The first contour 11 and the second contour 12 in the third image 30 may be represented by, for example, a solid line and a dashed line, respectively, to be distinguished from each other.

Unlike in FIG. 3, the first contour 11 and the second contour 21 may be represented in different colors to be distinguished from each other. The third image 30 may represent the difference between the first contour 11 and the second contour 21 in any of a variety of ways, and thus, the third image 30 is not limited to the above.

Therefore, the user may easily perceive the difference between the first contour 11 and the second contour 21 on the first image 30.

FIG. 4 illustrates a third image 30a displayed on the display apparatus 120 of FIG. 1, according to another embodiment of the present invention.

Referring to FIGS. 2 and 4, the first image 10 including the first contour 11 and the second image 20 including the second contour 21 are overlapped on the third image 30 so that a difference between the first contour 11 and the second contour 21 is represented on the third image 30a. A non-overlapping heart wall area between the first contour 11 and the second contour 21 in the third image 30a, i.e., a gap area GA, may be displayed to be distinguished from other areas. In some embodiments, the gap area GA in the third image 30a may be represented hatched or in a different color to be distinguished from the other areas. Therefore, the user may easily perceive the difference between the first contour 11 and the second contour 21 on the third image 30a.

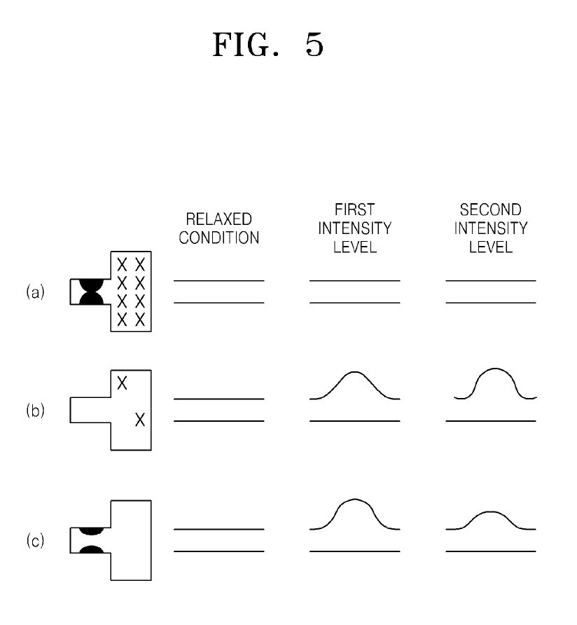

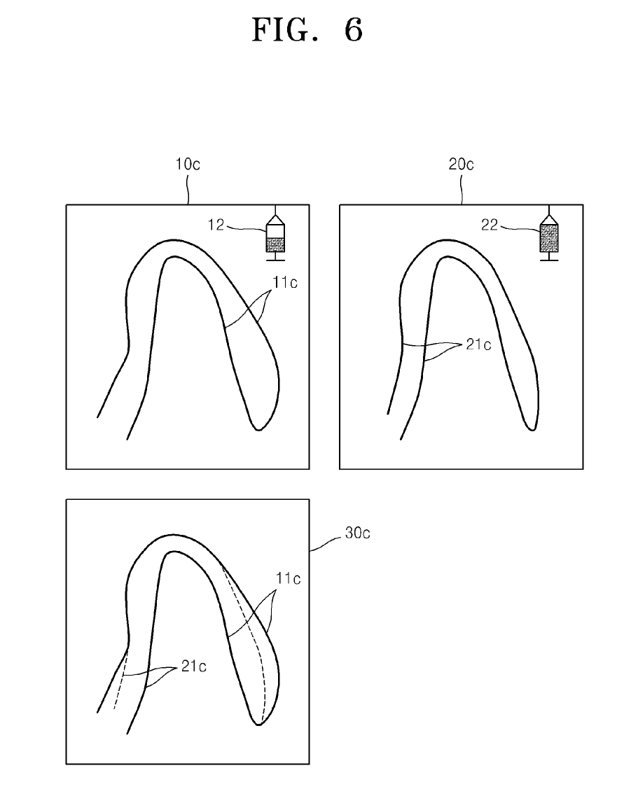

FIG. 5 is a view illustrating changes in thickness of a cardiac muscle according to types of heart conditions and stress levels. In particular, (A) to (C) in FIG. 5 illustrate different types of heart conditions of a subject observed to have changes in thickness of the cardiac muscle when the subject is in a relaxed state, is subjected to a first stress intensity level, or is subjected to a second intensity stress level.

(A) in FIG. 5 illustrates a heart condition with involving contraction of the coronary arteries of the subject and severe cardiac muscle damage. In this case, no change in thickness of the cardiac muscle was found in a relaxed state or when the subject is subjected to the first stress intensity level or the second stress intensity level.

(B) in FIG. 5 illustrates a heart condition with no contraction of the heart coronary arteries of the subject and nearly zero cardiac muscle damage. The thickness of the cardiac muscle increased when the subject is subjected to the first intensity stress level or the second stress intensity level higher than the first intensity stress level, as compared with the thickness of the cardiac muscle in a relaxed state.

(C) in FIG. 5 illustrates a heart condition with contraction of the coronary arteries of the subject and no cardiac muscle damage. The thickness of the cardiac muscle increased when the subject is subjected to the first stress intensity level or the second stress intensity level higher than the first stress intensity level, as compared with the thickness of the cardiac muscle in a relaxed state, with a greater increase in thickness of the cardiac muscle when the first stress intensity level lower than the second stress intensity level is applied, as compared with when the second stress intensity level is applied.

Therefore, whether the heart has an illness, such as cardiac muscle damage, contraction of the heart coronary arteries, or like, may be diagnosed by comparing changes in thickness of the cardiac muscle after subjecting the subject to stress at different intensity levels.

Referring back to FIGS. 1 to 3, the user may intuitively perceive the difference in heart wall thickness of the subject on the third image 30 that represents the difference between the first contour 11 and the second contour 21 displayed on the display apparatus 120. Therefore, the user may be convenienced in diagnosing the heart 210 of the subject 200, and a diagnosis speed may be improved. Since the third image 30 represents the difference between the first contour 11 and the second contour 21, this may ensure an objective comparison between the thicknesses of the heart wall when the subject 200 is subjected to stress at different intensity levels, and diagnosis accuracy may be increased.

If only the first image 10 and/or the second image 20, excluding the third image 30, are displayed on the display apparatus 120, the user may not be able to readily perceive a difference in heart wall thickness when the subject 200 is subjected to stress at different intensity levels. Thus, diagnosis accuracy may be reduced. To increase diagnosis accuracy, an additional task that, for example, the user reconfirms the first image 10 and/or the second image 20 may be performed. However, such an additional task may delay the diagnosis time.

Therefore, according to the above embodiments of the present invention, the diagnosis imaging apparatus 100 may provide user convenience to diagnose and may improve the diagnosis speed.

The display apparatus 120 may also display the first image 10 and the second image 20 along with the third image 30.

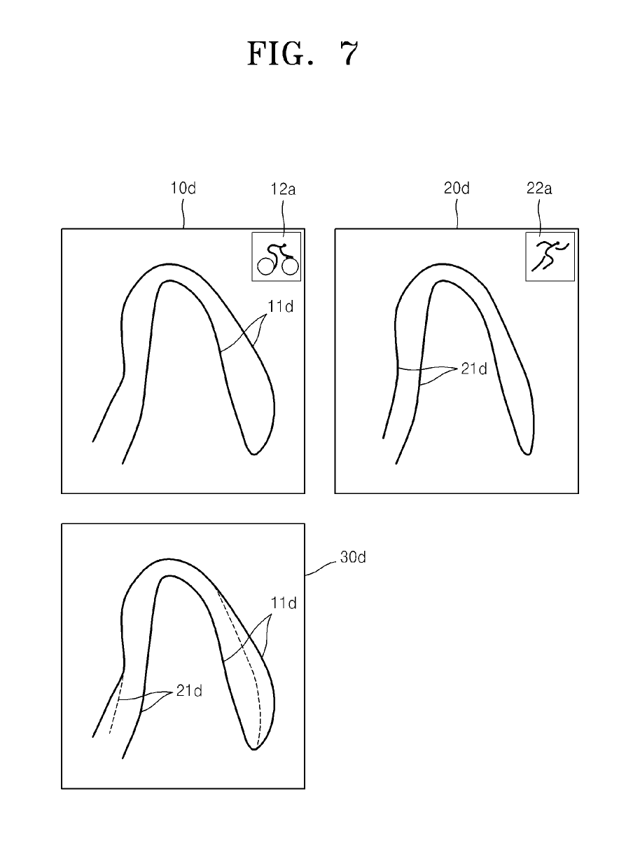

FIG. 6 illustrates first to third images 10c to 30c displayed on the display apparatus 120 of FIG. 1, according to another embodiment of the present invention.

Referring to FIG. 6, the display apparatus 120 (FIG. 1) may display the first image 10c and the second image 20c along with the third image 30c. The user may intuitively perceive a difference in thickness of the heart wall between when the subject is subjected to a first stress intensity level and when the subject is subjected to a second stress intensity level on the first to third images 10c to 30c.

The first image 10c and the second image 20c may further include a first marker 12 and a second marker 22, respectively, representing stress intensity levels the subject is subjected to, in addition to the first contour 11c and the second contour 21c. The first marker 12 and the second marker 22 may display the stress intensity level the subject is subjected to as at least one of an image and text. As illustrated in FIG. 6, the first marker 12 may represent the first stress intensity level by displaying an image of administration of a low drug dosage, and the second marker 22 may represent the second stress intensity level higher than the first intensity level, by displaying an image of administration of a large drug dosage.

The user may readily perceive the first image 10c corresponding to the first stress intensity level and the second image 20c corresponding to the second stress intensity level from the first marker 12 and the second marker 22.

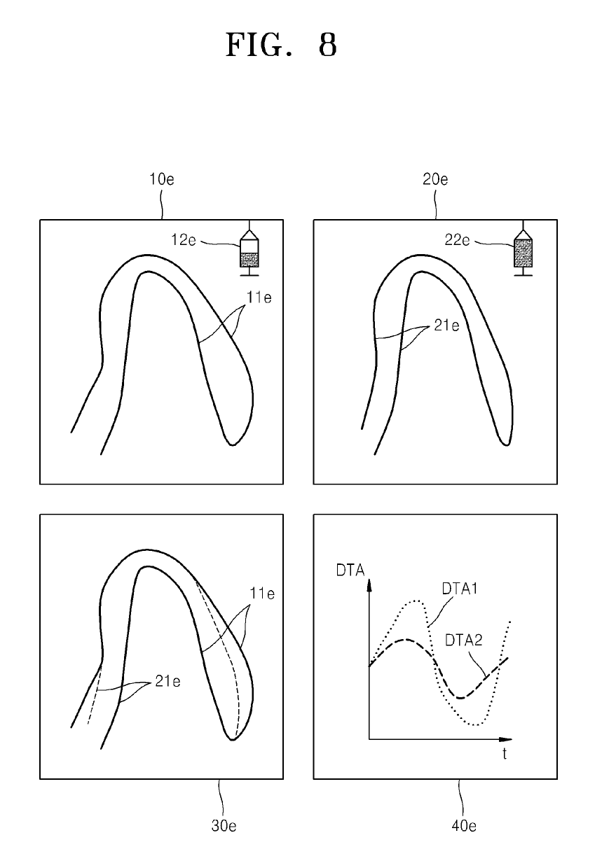

FIG. 7 illustrates first to third images 10d to 30d displayed on the display apparatus 120 of FIG. 1.

Referring to FIG. 7, the first image 10d and the second image 20d may include a first marker 12a and a second marker 22a, respectively, representing stress intensity levels the subject is subjected to. The first marker 12a may represent the first stress intensity level by displaying an image of riding a bike, and the second marker 22a may represent the second stress intensity level, higher than the first intensity level, by displaying an image of walking or running.

The first markers 12 and 12a and the second markers 22 and 22a in FIGS. 6 and 7 are only for illustrative purposes, not for limiting the embodiments thereof. The first markers 12 and 12a and the second markers 22 and 22a may display the stress intensity level the subject is subjected to in a variety of manners.

Referring back to FIGS. 1 and 3, the image processing apparatus 110 may acquire first data of the heart 210 of the subject 200 based on the first image data of the subject 200 acquired when the subject 200 is subjected to a first stress intensity level, and may acquire second data of the heart 210 based on the second image data acquired when the subject 200 is subjected to a second stress intensity level higher than the first stress intensity level. The image processing apparatus 110 may acquire a fourth image representing a difference between the first data and the second data. The display apparatus 120 may display the fourth image along with the first to third images 10 to 30.

The first data and the second data may be used to evaluate the functions of the heart 210. For example, the first data and the second data may be heart strain, a strain rate as strain with respect to time obtained by dividing strain by time, a heart wall thickness, a cardiac muscle change rate, or a heart volume, etc.

FIG. 8 illustrates first to fourth images 10e to 40e displayed on the display apparatus 120 of FIG. 1, according to another embodiment of the present invention.

Referring to FIG. 8, the display apparatus 120 (see FIG. 1) may display the fourth image 40e along with the first to third images 10e to 30e. The above-descriptions of the first to third images may be referred to herein to avoid redundancy.

The fourth image 40e may include a graph of first data DTA1 and second data DTA2 with respect to time t, wherein the time t may be a frame. The user may more easily analyze a difference between the first image 10e and the second image 20e from the fourth image 40e.

Although in FIG. 8 the fourth image 40e illustrates one piece of first data DAT1 and one piece of second data DTA2, the fourth image 40e may display a plurality of pieces of first data DAT1 and a plurality of pieces of second data DAT2. The first image 10e and the second image 20e may be represented with a plurality of pieces of first data DTA1 and a plurality of pieces of second data DTA2 that are assigned to a plurality of segments of the heart, respectively, as data for evaluating functions of the heart.

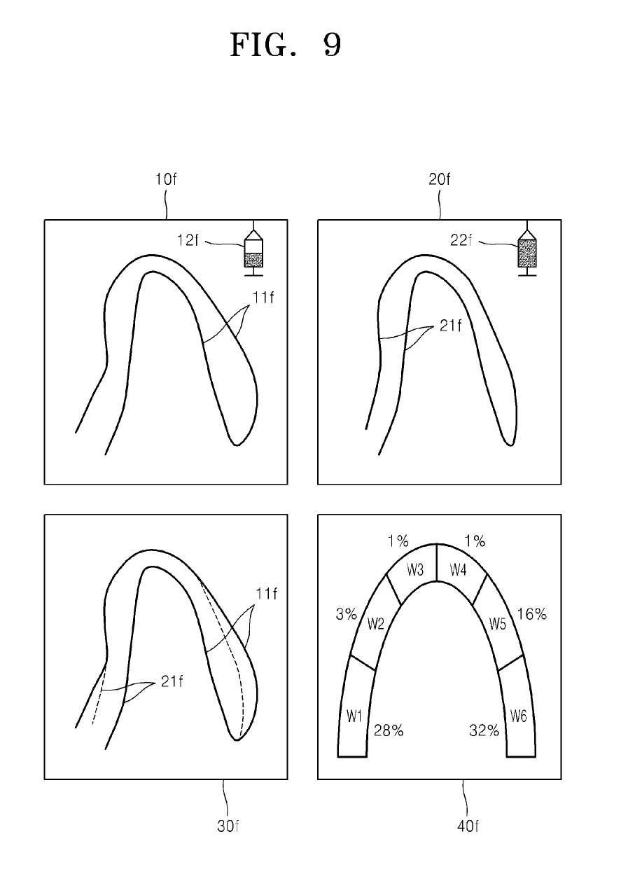

FIG. 9 illustrates first to fourth images 10f to 40f displayed on the display apparatus 120 of FIG. 1, according to another embodiment of the present invention.

Referring to FIG. 9, the fourth image 40f may represent a plurality of segments W1-W6 of the heart, each with a quantitative difference between the first data and the second data. In FIG. 9, the first data and the second data may be heart wall thicknesses. For example, the second segment W2 has a difference of about 3% between a heart wall thickness corresponding to the stress at the first intensity level and a heart wall thickness corresponding to the stress at the second intensity level higher than the first intensity level.

Further to the embodiments of FIGS. 8 and 9, a fourth image representing a difference between the first data corresponding to the first intensity level of stress and the second data corresponding to the second intensity level of stress may be displayed in any of a variety of manners depending on the types of the first and second data to evaluate functions of the heart.

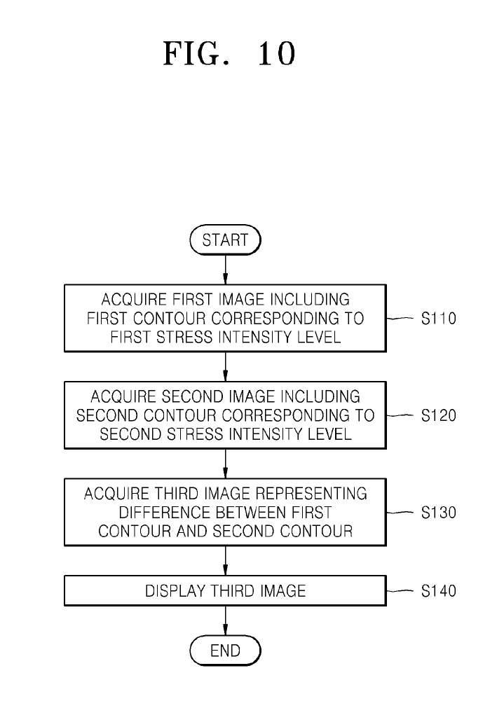

FIG. 10 is a flowchart of an operation method of a diagnosis imaging apparatus, according to an embodiment of the present invention.

Referring to FIG. 10, the diagnosis imaging apparatus may acquire a first image including a first contour of the heart wall based on first image data of the heart of the subject acquired when the subject is subjected to stress at a first intensity level (Operation S110). The diagnosis imaging apparatus may acquire a second image including a second contour of the heart wall based on second image data of the heart of the subject acquired when the subject is subjected to at a second stress intensity level higher than the first stress intensity level (Operation S120). The diagnosis imaging apparatus may acquire a third image representing a difference between the first contour and the second contour based on the first image and the second image (Operation S130). The diagnosis imaging apparatus may display the third image (Operation S140).

The operation method of FIG. 10 may be performed by the diagnosis imaging apparatus 100 of FIG. 1. The relevant descriptions described above with reference to FIGS. 1 to 9 may be referred to as a description of each operation of the method.

According to the embodiments described above, diagnosis imaging apparatuses and operating methods thereof are efficient.

The embodiments of the method described above may be written as computer programs and can be implemented in general-use digital computers that execute the programs using a computer-readable recording medium. Data used in the above-described embodiments can be recorded on a medium in various means. Examples of the computer-readable recording medium include magnetic storage media (e.g., ROM, RAM, USB, floppy disks, hard disks, etc.), optical recording media (e.g., CD-ROMs, or DVDs), and peripheral component interfaces (PCI) (e.g., PCI-express, or Wifi).

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A diagnosis imaging apparatus comprising:
an image processing apparatus for acquiring a first image including a first contour of a heart wall based on first image data of a heart of a subject acquired when the subject is subjected to a first stress intensity level, acquiring a second image including a second contour of the heart wall based on second image data of the heart of the subject acquired when the subject is subjected to a second stress intensity level, and acquiring a third image representing a difference between the first contour and the second contour based on the first image and the second image; and
a display apparatus for displaying the third image.

2. The diagnosis imaging apparatus of claim 1, wherein the third image represents the overlapping of the first contour with the second contour.

3. The diagnosis imaging apparatus of claim 2, wherein the display apparatus displays the first image and the second image along with the third image.

4. The diagnosis imaging apparatus of claim 2, wherein the first image and the second image each further comprise a marker indicating a stress intensity level the subject is subjected to.

5. The diagnosis imaging apparatus of claim 2, wherein the image processing apparatus acquires first data of the heart based on the first image data, second data of the heart based on the second image data, and a fourth image representing a difference between the first data and the second data; and the display apparatus displays the fourth image along with the first to third images.

6. The diagnosis imaging apparatus of claim 5, wherein the first data and the second data each include at least one of heart strain, a strain rate, a heart wall thickness, a cardiac muscle change rate, and a heart volume.

7. The diagnosis imaging apparatus of claim 6, wherein the fourth image includes a graph of the first data and the second data with respect to time.

8. The diagnosis imaging apparatus of claim 6, wherein the first data and the second data each has a plurality of pieces of data corresponding to a plurality of segments of the hearts.

9. The diagnosis imaging apparatus of claim 8, wherein the first image represents the plurality of segments of the heart each with a quantitative difference between the first data and the second data.

10. The diagnosis imaging apparatus of claim 1, wherein the stress at the first intensity level and the stress at the second intensity level are applied by administering different drug dosages to the subject, or by the subject exercising at different intensity levels.

11. An operation method of a diagnosis imaging apparatus, the method comprising:
acquiring a first image including a first contour of a heart wall based on first image data of a heart of a subject acquired when the subject is subjected to a first stress intensity level;
acquiring a second image including a second contour of the heart wall based on second image data of the heart of the subject acquired when the subject is subjected to a second stress intensity level;
acquiring a third image representing a difference between the first contour and the second contour based on the first image and the second image; and
displaying the third image.

12. The operation method of claim 11, further comprising:
acquiring first data of the heart based on the first image data;
acquiring second data of the heart based on the second image data;
acquiring a fourth image representing a difference between the first data and the second data; and
displaying the fourth image along with the third image.

13. The operation method of claim 12, wherein the first data and the second data each include at least one of heart strain, a strain rate, a heart wall thickness, a cardiac muscle change rate, and a heart volume.

14. A computer-readable recording medium having embodied thereon a program for executing the operation method of a diagnosis imaging apparatus according to claim 11.

15. A display apparatus displaying a third image representing a difference between a first contour and a second contour of a heart wall, the first contour of the heart wall being based on first image data of a heart of a subject acquired when the subject is subjected to a first stress intensity level, and the second contour being based on second image data of the heart of the subject acquired when the subject is subjected to a second stress intensity level.
